(19) United States
(12) Patent Application Publication
(10) Pub. No.: US 2007/0004014 A1
(43) Pub. Date: Jan. 4, 2007

(54) METHOD FOR PRODUCING L-THREONINE

(76) Inventors: Yuichiro Tsuji, Kawasaki-shi (JP); Naoto Kato, Kawasaki-shi (JP); Naoto Koyama, Kawasaki-shi (JP); Yuji Joe, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/427,482

(22) Filed: Jun. 29, 2006

Related U.S. Application Data
(60) Provisional application No. 60/695,846, filed on Jul. 5, 2005.

(30) Foreign Application Priority Data
Jun. 29, 2005 (JP) 2005-189106
Apr. 24, 2006 (JP) 2006-119334

(51) Int. Cl. C12P 13/04; C12N 9/12; C12N 15/74; C12N 1/21

(52) U.S. Cl. 435/106; 435/194; 435/252.33; 435/488

(57) ABSTRACT

A method for producing L-threonine is described which includes the steps of culturing a microorganism belonging to the genus Escherichia that has an ability to produce L-threonine, in a fermentation medium containing a carbon source, a nitrogen source, and a sulfur source, and collecting L-threonine, wherein the sulfur concentration in the medium is regulated so that it is a predetermined level or lower.

This application claims priority under 35 U.S.C. § 119(a) to JP2005-189106, filed in Japan on Jun. 29, 2005 and JP2006-119334, filed on Apr. 24, 2006, and also under 35 U.S.C. § 119(e) to U.S. Provisional Patent Application 60/695,846, filed Jul. 5, 2005, the entireties of which are incorporated by reference. The Sequence Listing on Compact Disk filed herewith is also hereby incorporated by reference in its entirety (File Name: US-236 Seq List; File Size: 38 KB; Date Created: Jun. 29, 2006).

### BACKGROUND OF THE INVENTION

1. Technical Field

The present invention relates to a technique for use in the fermentation industry. More specifically, the present invention relates to a method for efficiently producing L-threonine by fermentation utilizing an Escherichia bacterium. L-Threonine is one of the essential amino acids and is useful as an ingredient in nutritional mixtures for medical purposes. It is further utilized in various ways as an animal feed additive, and as a reagent in the pharmaceutical and chemical industries.

2. Background Art

L-amino acids such as L-threonine and L-isoleucine are industrially produced by fermentation using amino acid-producing bacteria, such as coryneform bacteria or Escherichia bacteria which have an ability to produce these L-amino acids. As these amino acid-producing bacteria, bacterial strains isolated from nature or artificial mutants of these bacterial strains are used. Recombinants of bacterial strains in which L-amino acid biosynthesis enzymes are enhanced by gene recombination and so forth are used in order to improve the productivity.

Specifically, mutant strains of L-threonine producing Escherichia bacteria are known, such as a 6-dimethylaminopurine resistant strain (Japanese Patent Laid-open (Kokai) No. 5-304969) and a borrelidin resistant strain (International Patent Publication WO98/04715). Methods of using recombinant Escherichia bacteria to produce L-threonine are known, specifically using a strain in which the threonine operon is amplified by using a plasmid (U.S. Pat. No. 5,175,107), or in which the phosphoenolpyruvate carboxylase gene and the aspartase gene are amplified by using a plasmid (U.S. patent application Ser. No. 2002/0110876).

As genes coding for enzymes involved in the biosynthesis of L-threonine in Escherichia coli, the aspartokinase III gene (lysC), aspartate semialdehyde dehydrogenase gene (asd), aspartokinase I-homoserine dehydrogenase gene (thrA), homoserine kinase gene (thrB) and threonine synthase gene (thrC) and so forth are known. The thrA, thrB and thrC (thrABC) constitute the threonine operon. The threonine operon forms an attenuator structure, and the expression thereof is inhibited by isoleucine and threonine in a culture medium. It is known that the fermentation yield is improved when the leader sequence of this attenuation region or the attenuator is removed from the threonine operon (U.S. Pat. No. 5,538,873, Biotechnology Letters, Vol. 24, No. 21, November 2002, and International Patent Publication WO05/049808).

To date, methods for producing L-threonine which have been developed include use of a batch culture using a fermenter initially containing all the nutrients, a fed-batch culture using a fermenter initially containing predetermined nutrients and continuously fed with one or more additional nutrients (U.S. Pat. No. 5,538,873and European Patent No. 593792), and a method of regulating the saccharide concentration so that it is maintained at a predetermined level or lower (International Patent Publication WO05/014840 and International Patent Publication WO05/014843). Furthermore, another method for producing L-threonine which has been developed is a method of feeding a culture medium, or adding nutrients to a medium, so that phosphoric acid and carbon sources serve as growth limiting factors (U.S. Pat. No. 5,763,230).

Sulfur is an essential factor for bacterial cell growth and is usually included in the medium for L-threonine fermentation in the form of ammonium sulfate. With regard to L-threonine production by fermentation; however, regulation of the sulfur concentration in the fermentation medium and the effect of lowering the sulfur concentration are unknown.

### SUMMARY OF THE INVENTION

An object of the present invention is to provide a method for efficiently producing L-threonine by using an Escherichia bacterium having an ability to produce L-threonine.

The inventors of the present invention assiduously studied in order to achieve the foregoing object. As a result, the inventors found that the sulfur concentration in the medium could affect fermentation results, and that the yield of L-threonine during fermentation can be improved by regulating the sulfur concentration in the fermentation medium so that it is maintained at a predetermined level or lower, and thus accomplished the present invention.

It is an object of the present invention to provide a method for producing L-threonine comprising culturing a microorganism belonging to the genus Escherichia that has an ability to produce L-threonine, in a fermentation medium containing a carbon source, a nitrogen source, and a sulfur source, and collecting L-threonine from the culture, wherein the sulfur concentration in the medium is regulated so that it is at a predetermined level or lower.

It is a further object of the present invention to provide the method as described above, wherein the sulfur concentration in the fermentation medium is regulated so that it is 0.35 g/L or lower.

It is a further object of the present invention to provide the method as described above, wherein the microorganism is Escherichia coli.

It is a further object of the present invention to provide the method as described above, wherein an L-threonine biosynthesis enzyme in the microorganism is modified so that the enzyme is not subject to feedback inhibition by L-threonine.

It is a further object of the present invention to provide the method as described above, wherein the L-threonine biosynthesis enzyme is selected from the group consisting of aspartokinase, homoserine kinase, threonine synthase, and combinations thereof.

It is a further object of the present invention to provide the method as described above, wherein the sulfur source is selected from the group consisting of sulfates, thiosulfates, sulfites, cysteine, cystine, glutathione, and combinations thereof.

It is a further object of the present invention to provide the method as described above, wherein the culture method is selected from the group consisting of a batch culture method, a fed-batch culture method, and a continuous culture method.

It is a further object of the present invention to provide the method as described above, wherein the culture method is a fed-batch culture method or a continuous culture method, wherein a feed medium containing a sulfur source is added to the culture in a fermenter.

It is a further object of the present invention to provide the method as described above, wherein said feed medium further contains a carbon source and a nutrient having a growth promoting effect, and wherein said feed medium is added to the fermenter continuously or intermittently so that concentration of the carbon source in the culture medium is maintained at 30 g/L or lower after the end of the logarithmic growth of the microorganism.

It is a further object of the present invention to provide a method for producing animal feed additive based on fermentation broth comprising:

A) culturing a microorganism belonging to the genus Escherichia that has an ability to produce L-threonine, in a fermentation medium containing a carbon source, a nitrogen source, and a sulfur source,

B) maintaining the sulfur concentration in the medium during the fermentation at a predetermined level or lower,

C) drying the crude fermentation broth so that the water content is 10% or less by weight.

### BRIEF DESCRIPTION OF THE DRAWING

FIG. 1 shows the relationship between the amount of sulfur initially added to a medium, and the L-threonine yield.

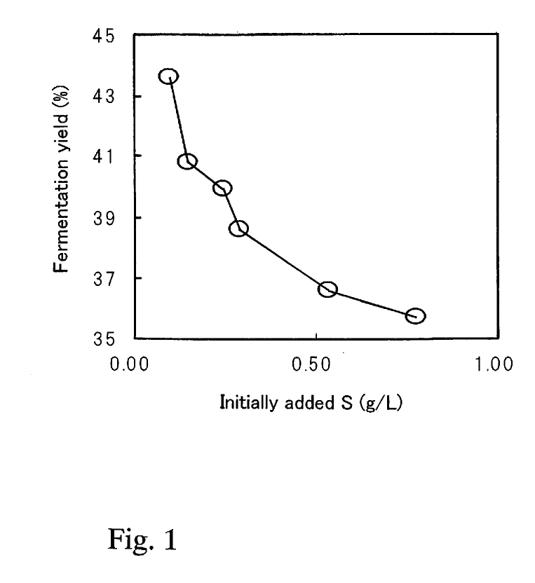

### DETAILED DESCRIPTION OF THE PREFERRED EMBODIMENTS

<1> The Method of the Present Invention

The method of the present invention is a method for producing L-threonine by culturing a microorganism belonging to the genus Escherichia that has an ability to produce L-threonine, in a fermentation medium containing a carbon source, a nitrogen source, and a sulfur source so that L-threonine is produced in the medium, wherein the sulfur concentration in the medium is regulated so that it is maintained at a predetermined level or lower. In the present invention, the term “sulfur concentration” means the concentration of the sulfur source in terms of sulfur atom concentration.

The medium used for the present invention may be any medium so long as it is a liquid medium which contains a carbon source, a nitrogen source, and a sulfur source as nutrients, and the medium is not particularly limited except that it is adjusted so that the sulfur concentration is maintained at a predetermined level or lower. The sulfur source may be any sulfur-containing source. Salts of sulfuric acid, such as sulfates, thiosulfates, and sulfites, as well as sulfur-containing amino acids such as cysteine, cystine, and glutathione are desirable. Among these, ammonium sulfate is particularly preferable. The salts are not particularly limited, and ammonium salts, calcium salts, sodium salts, potassium salts, magnesium salts, manganese salts, and iron salts may be used. Furthermore, the medium may contain only one type, or two or more types, of these substances. The concentration indicated by the phrase “predetermined level or lower” may be any concentration at which the L-threonine yield is improved as compared with that in a medium containing a large amount of sulfur, or a conventional fermentation medium. Specifically, the concentration of sulfur in the fermentation medium is preferably 0.35 g/L or lower, more preferably 0.25 g/L or lower, particularly preferably 0.10 g/L or lower. One of the characteristics of the present invention is that the sulfur concentration in the fermentation medium is regulated.

For the method of the present invention, a batch culture, fed-batch culture, and/or a continuous culture can be employed, and the sulfur concentration in the medium may be regulated to be at a predetermined level or lower in the initial medium, or limited to be at a predetermined level or lower by controlling the sulfur concentration in the feed medium or by using these techniques in combination. The same sulfur source may be used for the initial medium and the feed medium, or the sulfur source in the feed medium may be different from that used in the initial medium.

In the present invention, a fed-batch culture refers to a culture method wherein the medium is added, continuously or intermittently, into a vessel during the culture, and not removing the medium from the vessel before completion of culture. The continuous culture refers to a method of continuously or intermittently adding a medium into a vessel during the culture, and withdrawing the medium from the vessel (normally, in an amount equivalent to the medium-fed). The term “initial medium” means a medium used for batch culture before the feed medium is added in the fed-batch culture or continuous culture, and the term “feed medium” means a medium to be supplied to a fermenter when fed-batch culture or continuous culture is performed. The feed medium may contain all or a part of the components necessary for the growth of a microorganism. In the present invention, the term “fermentation medium” means a medium contained in a fermenter, and L-threonine is collected from this fermentation medium. Furthermore, in the present invention, the term “fermenter” means a vessel in which L-threonine fermentation is performed, and the shape thereof is not limited. A fermentation tank or a jar fermenter may be used. Furthermore, the volume of the fermenter is not limited so long as L-threonine can be produced and collected.

Although the sulfur concentration is preferably limited to a predetermined level or lower throughout the whole culture process, it may be limited during just a part of the process. For example, when the method of the present invention includes a stage at which cells proliferate (growth phase) and a stage at which L-threonine is produced (L-threonine production phase), it is sufficient that the sulfur concentration is limited to a predetermined level or lower in the L-threonine production phase. In the growth phase (during cell proliferation), sulfur may be present in the medium at more than the predetermined amount, or the sulfur concentration may be limited to the predetermined level or lower. Furthermore, during the stage at which L-threonine is produced, the sulfur content does not need to be in the aforementioned range throughout the whole period of this stage, and the sulfur content may be at the aforementioned level or higher during an early period of that stage, and may be reduced with the culture time. Furthermore, sulfur may be added intermittently when it runs short. The term “growth phase” used in the present invention means a period within 3 hours, preferably 6 hours, particularly preferably 10 hours, from the start of the culture, during which period the carbon source is primarily consumed for the bacterial cell growth, that is, cells logarithmically proliferate. The term “L-threonine production phase” used in the present invention means a period after the initial 3 hours, preferably 6 hours, particularly preferably 10 hours, from the start of culture, during which the carbon source is primarily consumed for L-threonine production.

It is sufficient that the fermentation medium contains a minimum amount of sulfur which is required for the growth of the microorganism; however, the amount of sulfur may temporarily run short. The phrase “run(s) short” means that the sulfur concentration is reduced compared to earlier points in the culture, and may even become “0”. The term “temporarily” means that, for example, sulfur may run short for a period corresponding to about 20%, about 40%, or about 60% at most, of the whole fermentation period. During the period when sulfur runs short, although the concentration may temporarily be 0, sulfur is desirably present in the fermentation medium at a concentration of 1 μg/L or more, 10 μg/L or more, or 100 μg/L or more. Thus, even if the sulfur concentration temporarily becomes 0, a culture of a medium containing sulfur for any period is encompassed within the expression “culturing a microorganism belonging to the genus Escherichia in a fermentation medium containing a carbon source, a nitrogen source, and a sulfur source”. The sulfur concentration in the culture medium can be measured by the ion chromatography method and the hot flask method.

Furthermore, according to the present invention, the sulfur concentration in the feed medium may be adjusted so that it is limited to a predetermined level or lower when a fed-batch culture is used. For example, when the sulfur concentration is limited by using a fed-batch culture, it is preferable to control the sulfur concentration in the fermentation medium to be 0.35 g/L or lower, desirably 0.25 g/L or lower, more desirably 0.10 g/L or lower.

Examples of the carbon source used in the present invention include saccharides such as glucose, glycerol, fructose, sucrose, maltose, mannose, galactose, starch hydrolysate, and molasses. Glucose and sucrose are particularly preferred. In addition, organic acids such as acetic acid and citric acid, and alcohols such as ethanol can also be used alone or in combination with another carbon source. Furthermore, the raw material of the carbon source may be cane molasses, beet molasses, high test molasses and citrus molasses, and hydrolysates of natural raw materials such as cellulose, starch, corn, cereal and tapioca. Furthermore, carbon dioxide dissolved in a culture medium can also be used as a carbon source. These carbon sources can be used in the initial medium and/or in the feed medium. The medium may contain one or more types of these carbon sources. Furthermore, the same carbon source may be used for the initial medium and the feed medium, or the carbon source of the feed medium may be different from that of the initial medium. For example, glucose may be used in the initial medium, while sucrose may be used in the feed medium.

Examples of the nitrogen source used in the present invention include ammonia, ammonium salts such as ammonium sulfate, ammonium carbonate, ammonium chloride, ammonium phosphate, ammonium acetate and urea, nitrates, and so forth. Ammonia gas and aqueous ammonia which are used to adjust the pH can also be utilized as the nitrogen source. Furthermore, peptone, yeast extract, meat extract, malt extract, corn steep liquor, soybean hydrolysate, and so forth can also be utilized. The medium may contain one or more types of these nitrogen sources. These nitrogen sources can also be used in the initial medium and/or in the feed medium. Furthermore, the same nitrogen source can be used in both the initial medium and in the feed medium, and the nitrogen source of the feed medium may be different from that of the initial medium.

Furthermore, the medium of the present invention preferably contains a phosphoric acid source in addition to a carbon source, a nitrogen source, and sulfur. As the phosphoric acid source, potassium dihydrogenphosphate, dipotassium hydrogenphosphate, and phosphate polymers such as pyrophosphoric acid can be utilized.

Furthermore, the medium of the present invention may contain a growth promoting factor (a nutrient having a growth promoting effect) in addition to a carbon source, a nitrogen source, and sulfur. Growth promoting factors which can be used include trace metals, amino acids, vitamins, fatty acids, nucleic acids, as well as peptone, casamino acid, yeast extract, and soybean protein hydrolysate.

Examples of the trace metals include iron, manganese, magnesium, calcium, potassium, sodium, and so forth. Examples of the vitamins include vitamin B₁, vitamin B₂, vitamin B₆, nicotinic acid, nicotinic acid amide, vitamin B₁₂, and so forth. These growth promoting factors may be contained in the initial medium or in the feed medium.

Furthermore, when an auxotrophic mutant that requires an amino acid or the like for growth thereof is used, it is preferable to add the required nutrient to the medium. In particular, since the L-threonine biosynthetic pathway is often enhanced and the L-threonine degrading ability is often attenuated in the L-threonine producing bacteria that can be used for the present invention as described below, L-lysine, L-homoserine, L-isoleucine, L-methionine, or a combination thereof are preferably added.

The initial medium and the feed medium may have the same or different compositions. Furthermore, the initial medium and the feed medium may have the same or different sulfur concentration. Furthermore, when the feed medium is added at multiple stages, the composition of the feed medium added at each stage may be the same or different.

The culture is preferably performed with aeration, preferably at a fermentation temperature of 20 to 45° C., particularly preferably at 33 to 42° C. The oxygen concentration is preferably adjusted to 5 to 50%, more preferably to about 10%. Furthermore, aeration is preferably performed with the pH adjusted to 5 to 9. If the pH is lowered during the culture, for example, calcium carbonate or an alkali such as ammonia gas and aqueous ammonia may be added to neutralize the culture. When the culture is performed under such conditions, preferably for about 10 to 120 hours, a marked amount of L-threonine accumulates in the culture medium. The concentration of accumulated L-threonine is not limited so long as L-threonine can be isolated and collected from the medium, and it is 50 g/L or higher, desirably 75 g/L or higher, more desirably 100 g/L or higher.

In the present invention, culture of the microorganism may be performed by seed culture and/or a main culture in order to induce accumulation of L-threonine higher than a certain level. The seed culture may be performed by shaking, using a flask or the like, or a batch culture. The main culture may be performed as fed-batch culture or a continuous culture. Alternatively, both the seed culture and the main culture may be performed as batch culture.

In these culture methods, when the L-threonine concentration reaches a predetermined level, some of the fermentation broth may be withdrawn, and fresh medium may be added to repeat the culture. As the fresh medium, a medium containing a carbon source and a nutrient having a growth promoting effect (growth promoting factor) is preferred, and it preferably contains sulfur at a predetermined concentration or lower. The expression “predetermined concentration or lower” means that the medium to be added is adjusted so that the sulfur concentration in the fermentation medium becomes 0.35 g/L or lower, desirably 0.25 g/L or lower, more desirably 0.10 g/L or lower. As the carbon source, glucose, sucrose, and fructose are preferred. As the growth promoting factor, nitrogen sources, phosphoric acid, amino acids and so forth are preferred. As the nitrogen source, ammonia, ammonium salts such as ammonium sulfate, ammonium carbonate, ammonium chloride, ammonium phosphate, ammonium acetate and urea, nitrates, and so forth can be used. Furthermore, as the phosphoric acid source, potassium dihydrogenphosphate and dipotassium hydrogenphosphate can be used. As for amino acids, when an auxotrophic mutant strain is used, it is preferable to supplement with the required amino acid.

When fed-batch culture or continuous culture is performed according to the present invention, the addition of the feed medium may be intermittently suspended, so that the supply of saccharide or nutrients is temporarily suspended. The addition of the feed medium is preferably suspended for, at the most, 30%, desirably 20%, particularly desirably 10%, of the feeding time. The “feeding time” means the period from the beginning of the first addition of the feed medium to end of the last addition of the feed medium. The suspension of the addition of feed medium can be less than 30%, and most preferably, less than 10%. When the feed medium is intermittently added, the feed medium may be initially added over a predetermined time, and the second and following additions may be controlled so that it is started when an increase in the pH or the dissolved oxygen concentration is detected by a computer. Such detection typically occurs upon depletion of the carbon source in the fermentation medium during an addition/suspension period prior to a certain addition period, and thus the substrate concentration in the culture tank should be automatically and consistently maintained at a low level (U.S. Pat. No. 5,912,113).

The feed medium used for the fed-batch culture is preferably a medium containing a carbon source and a nutrient having a growth promoting effect (growth promoting factor), and may contain sulfur so that the sulfur concentration in the fermentation medium is maintained at a predetermined concentration or lower. The expression “predetermined concentration or lower” as used herein means that the added medium is adjusted so that the sulfur concentration in the fermentation medium is maintained at a concentration of 0.35 g/L or lower, desirably 0.25 g/L or lower, more desirably 0.10 g/L or lower. Although the sulfur concentration of the feed medium itself may be within or out of the aforementioned concentration rage, it is preferably within the aforementioned concentration range.

As the carbon source, glucose, sucrose, and fructose are preferred. As the growth promoting factor, nitrogen sources, phosphoric acid, amino acids and so forth are preferred. As the nitrogen source, ammonia, ammonium salts such as ammonium sulfate, ammonium carbonate, ammonium chloride, ammonium phosphate, ammonium acetate and urea, nitrates, and so forth can be used. Furthermore, as the phosphoric acid source, potassium dihydrogenphosphate and dipotassium hydrogenphosphate can be used. As for the amino acids, when an auxotrophic mutant strain is used, it is preferable to supplement with the required amino acid. Furthermore, the feed medium may be of one type, or a mixture of two or more types of media. When two or more types of feed media are used, the media may be mixed and added by using one feed can, or by using two or more feed cans.

Furthermore, when a fed-batch culture is performed, the addition is preferably performed so that the amount of saccharide does not exceed 30 g/L, preferably 20 g/L, more preferably 10 g/L, of the whole fed-batch culture medium or fermentation medium. In particular, the saccharide concentration is preferably controlled to be within the aforementioned concentration range after the completion of the logarithmic growth of the microorganism. The feeding rate of the carbon source can be controlled by the method described in U.S. Pat. No. 5,912,113. Furthermore, the saccharide and phosphoric acid are preferably fed so that the concentrations are such that the saccharide and phosphoric acid are limiting factors for the bacterial cell growth. Phosphoric acid is present in the feed medium so that the phosphorous/carbon (P/C) ratio is 2 or lower, preferably 1.5 or lower, more preferably 1 or lower (U.S. Pat. No. 5,763,230).

When the continuous culture method is used for the present invention, the medium may be withdrawn and added simultaneously, or a part of the medium may be extracted, and then the medium may be added. Furthermore, the method may also be a continuous culture method of removing the culture medium containing L-threonine and bacterial cells and recycling only the cells to the fermenter (French Patent No. 2669935). As the method of continuously or intermittently adding a nutrient source, the same method as used in the fed-batch culture is used.

When the culture medium is intermittently removed, a part of L-threonine is collected when the L-threonine concentration reaches a predetermined level, and fresh medium is added to continue the culture. Furthermore, as for the amount of the fresh medium to be added, the final amount of the total medium in the culture after the addition of the fresh medium is equal to the amount of the culture medium before removal. The term “equal” as used herein means about 93 to 107% of the amount of the culture medium before the extraction.

When the culture medium is continuously withdrawn, the withdrawal is preferably started at the same time as, or after, the addition of the nutrient medium. For example, the starting time is, at maximum, 5 hours, preferably 3 hours, more preferably 1 hour, after the start of the addition. Furthermore, the amount of the culture medium which is withdrawn is preferably equal to the amount of the medium added.

The continuous culture method of reusing bacterial cells is a method of intermittently or continuously withdrawing the fermentation medium when the amino acid concentration reaches a predetermined level, withdrawing only L-threonine, and re-circulating filtration residues containing bacterial cells in the fermenter, and it can be performed by referring to, for example, French Patent No. 2669935.

Analysis of L-threonine and other amino acids can be performed by anion exchange chromatography followed by ninhydrin derivation as described in Spackman et al.(Analytical Chemistry 30:1190-1206 (1958)) or by reverse phase HPLC as described in Lindroth et al. (Analytical Chemistry 51:1167-1174).

<2> The Method for Producing an Animal Feed Additive from Fermentation Broth

An animal feed additive can be prepared from the fermentation broth by the following separation method.

L-threonine separation methods such as centrifuging, filtering, decanting flocculating or a combination of these can be used to remove or reduce biomass.

The broth obtained by this invention can be thickened or concentrated using known methods such as a rotary evaporator, thin layer evaporator, reverse osmosis, or nanofiltration. (see, for example, FR8613346B, U.S. Pat. No. 4,997,754, EP410005B, JP1073646B).

The concentrated broth is then processed by freeze-drying, spray-drying, spray granulation or any other process to give a preferably free flowing, finely divided powder, which can be used as an animal feed additive. This free-flowing finely divided powder can be converted into a coarse-grained, very free flowing, stable and largely dust-free product by using suitable compacting or granulating processes. Altogether, more than 90% of water is removed in this way so that the water concentration of the animal feed additive is less than 10%, preferably less than 5% by weight.

The protein content of the feed additive can be less than 10%, preferably less than 5% by weight, and the concentration of L-threonine can be more than 50%, preferably more than 85%, more preferably more than 95%. (see, for examples, U.S. Pat. No. 5,431,933, JP1214636B, U.S. Pat. Nos. 4,956,471, 4,777,051, 4,946,654, 5,840,358, 6,238,714, U.S. 2005/0025878)

The separation step described above does not necessarily have to be performed, but may be combined in a technically expedient manner.

<3> Escherichia Bacteria that can be Used for the Present Invention

Escherichia bacteria that can be used for the present invention are Escherichia bacteria having an ability to produce L-threonine, and the term “ability to produce L-threonine” used in the present invention means an ability, when the bacteria are cultured in a medium, of producing free L-threonine in the medium, that is, outside the cells, to such an extent that L-threonine can be collected from the medium. Preferably, the Escherichia bacterium used for the present invention is a strain which is bred so that the strain can produce a higher amount of L-threonine as compared with wild-type strains or parental strains. Specifically, it is preferable that the Escherichia bacterium produces an amount of L-threonine of 30 g/L or higher, more preferably 50 g/L or higher, particularly preferably 75 g/L or higher, using a typical culture method in which the sulfur concentration is not adjusted.

Parent strains of Escherichia bacteria used to obtain the Escherichia bacteria suitably used for the present invention are not particularly limited, and specific examples thereof include those mentioned in the work of Neidhardt et al. (Neidhardt, F.C. et al., Escherichia coli and Salmonella Typhimurium, American Society for Microbiology, Washington D.C., 1029, Table 1). Among these, for example, Escherichia coli is preferably used. Specific examples of the Escherichia coli include Escherichia coli W3110 (ATCC 27325) and Escherichia coli MG1655 (ATCC 47076), both of which are derived from a prototype wild-type strain K12, and so forth.

As for obtaining these strains, they can be obtained from, for example, American Type Culture Collection (address: P.O. Box 1549 Manassas, Va. 20108, United States of America). Each bacterial strain is given a corresponding registration number, and each strain can be ordered by using this registration number. The registration numbers corresponding to bacterial strains are listed in the catalogue of American Type Culture Collection.

<3-1> Impartation of L-threonine Producing Ability

Hereinafter, the method for imparting an L-threonine producing ability to an Escherichia bacterium will be described.

To impart an L-threonine producing ability, methods can be used which have been conventionally adopted for breeding Escherichia bacteria or coryneform bacteria, such as acquisition of an auxotrophic mutant, analogue resistant strain, or metabolism control mutant strain, all of which have an L-threonine producing ability, as well as construction of a recombinant strain in which an activity of an L-threonine biosynthesis enzyme is enhanced. For example, a mutant or recombinant strain can be modified so that an L-threonine biosynthesis enzyme is not subject to feedback inhibition, or a recombinant strain can be modified to enhance expression of an L-threonine biosynthesis enzyme gene. In the breeding of L-threonine producing bacteria by these methods, properties such as auxotrophy, analogue resistance, and a metabolic control mutation may be imparted either singly or in combination.

When activity of an L-threonine biosynthesis enzyme is enhanced, one or more of these enzymes' activities may be enhanced. Furthermore, imparting the properties mentioned above and enhancing enzyme activity, as mentioned above, may be implemented in combination.

An example of a method for imparting L-threonine producing ability to an Escherichia bacterium, or enhancing the L-threonine producing ability by enhancing activity of an L-threonine biosynthesis enzyme, will be explained below. An enzymatic activity can be enhanced by, for example, introducing a mutation into a gene coding for the enzyme or amplifying the gene so that intracellular activity of the enzyme increases. These can be achieved by utilizing gene recombination techniques.

Examples of the genes which encode L-threonine biosynthesis enzymes include the aspartokinase III gene (lysC), aspartate semialdehyde dehydrogenase gene (asd), aspartokinase I gene included in the thr operon (thrA, nucleotide numbers 337 to 2799 of SEQ ID NO: 1), homoserine kinase gene (thrB, nucleotide numbers 2801 to 3733 of SEQ ID NO: 1), and threonine synthase gene (thrC, nucleotide numbers 3734 to 5020 of SEQ ID NO: 1). Shown in the parentheses are the abbreviated names of these genes. Two or more of these genes may be introduced into a bacterium. L-threonine biosynthetic genes may be introduced into an Escherichia bacterium in which threonine degradation is suppressed. Examples of the Escherichia bacterium in which threonine degradation is suppressed include the TDH6 strain, which is deficient in threonine dehydrogenase activity (EP1149911A), and so forth.

Enzymatic activities of L-threonine biosynthesis enzymes are inhibited by L-threonine as the final product. Therefore, to construct an L-threonine producing bacterium, modifying L-threonine biosynthesis system genes so that the encoded enzymes are not subject to feedback inhibition by L-threonine is beneficial. The aforementioned thrA, thrB, and thrC genes constitute the threonine operon, and the threonine operon has an attenuator structure. Expression of the threonine operon is inhibited by isoleucine and threonine present in the culture medium, and is suppressed by attenuation. The aforementioned modification can be achieved by removing the leader sequence in the attenuation region (SEQ ID NO: 6), or by removing the attenuator (SEQ ID NO: 1) (International Patent Publication WO02/26993; Biotechnology Letters, Vol. 24, No. 21, November 2002; International Patent Publication WO05/049808, WO98/04715, WO03/097839). In particular, it is desirable to modify the threonine operon so that the sequence of the nucleotides 188 to 310 is removed from the sequence of SEQ ID NO: 1. Alternatively, modifying the threonine operon so that the sequence of the nucleotides 148 to 310 is removed from the sequence of SEQ ID NO: 1 is also desirable.

A unique promoter exists in an upstream region of the threonine operon, and it may be replaced with a non-native promoter (refer to WO98/04715). Alternatively, the threonine operon may be constructed so that expression of the genes involved in threonine biosynthesis is regulated by the repressor and promoter of lambda phage (refer to European Patent No. 0593792). Furthermore, an Escherichia bacterium modified so that it is not subject to feedback inhibition by L-threonine can also be obtained by selecting a strain resistant to α-amino-β-hydroxyvaleric acid (AHV).

Furthermore, multiple copies of the threonine operon which has been modified so that it is not subject to feedback inhibition by L-threonine preferably can be present. Alternatively, the operon is ligated to a strong promoter so that expression is increased. The copy number of the L-threonine operon can be increased by amplifying it using a plasmid and/or transferring it onto a chromosome using a transposon, Mu-phage, or the like (U.S. Pat. No. 5,175,107).

Furthermore, an aspartokinase III gene (lysC) which has been modified so that it is not subject to feedback inhibition by L-lysine is preferably used. Such a lysC gene can be obtained by the method described in U.S. Pat. No. 5,932,453.

In addition to the L-threonine biosynthesis enzymes, it is also preferable to enhance expression of genes relating to the glycolytic pathway, TCA cycle, or respiration chain, genes that regulate expression of a gene, and saccharide uptake genes. Examples of the genes that have an effect on L-threonine production include the transhydrogenase (pntAB) gene (European Patent No. 733712), phosphoenolpyruvate carboxylase gene (pepC) (International Patent Publication WO95/06114, US2005-0136518), phosphoenolpyruvate synthase gene (pps) (European Patent No. 877090), and pyruvate carboxylase gene of coryneform or Bacillus bacteria (International Patent Publication WO99/18228; European Patent Publication No. 1092776).

Furthermore, it is also preferable to enhance expression of a gene that imparts L-threonine resistance, or a gene that imparts L-homoserine resistance, or both in combination to a host. Examples of a gene that imparts resistance include the rhtA gene (Res. Microbiol., 2003 March, 154(2): 123-35), rhtB gene (European Patent Publication No. 0994190), rhtC gene (European Patent Publication No. 1013765), yfiK gene, and yeaS gene (European Patent Publication No. 1016710). Furthermore, the methods for imparting L-threonine resistance to a host are described in European Patent Publication No. 0994190 and International Patent Publication W090/04636.

Activities of enzymes encoded by the aforementioned genes can be increased by enhancing expression of the genes, for example, by increasing the copy numbers of the genes in the cells using gene recombination techniques. For example, a DNA fragment containing a target gene can be ligated to a vector that functions in a host microorganism, preferably a multi-copy type vector, and the host microorganism can be transformed with this vector DNA.

When an Escherichia coli gene is used as the target gene, it can be obtained by preparing primers by referring to the known gene sequence in Escherichia coli MG1655 or W3110 registered at GenBank and performing a polymerase chain reaction (PCR) using chromosomal DNA of Escherichia coli as a template (refer to White, T. J. et al., Trends Genet. 5, 185 (1989)). The target genes of other microorganisms can also be obtained from chromosomal DNAs or chromosomal DNA libraries of the microorganisms by PCR using primers prepared based on the known gene information for the microorganisms or sequence information from GenBank, or hybridization using an oligonucleotide prepared based on the aforementioned sequence information as a probe. Chromosomal DNA can be prepared from a donor microorganism, for example, by the method of Saito and Miura (refer to H. Saito and K. Miura, Biochem. Biophys. Acta, 72, 619 (1963); Text for Bioengineering Experiments, Edited by the Society for Bioscience and Bioengineering, Japan, pp. 97-98, Baifukan, 1992), and so forth.

Furthermore, since nucleotide sequences of genes coding for a target enzyme may vary depending on the species of Escherichia bacteria or bacterial strains, the target gene used for the present invention is not limited to known genes or gene sequences registered at GenBank, and may be a mutant or artificially modified gene. Such a mutant or modified gene may code for a protein which has a sequence that includes substitutions, deletions, insertions, additions, or the like, of one or several amino acid residues at one or more sites, so long as the function of the encoded target protein, that is, the L-threonine production ability, can be improved by amplification of the gene. The number meant by the term “several” used herein varies depending on positions of amino acid residues in the three-dimensional structure of the protein and types of the amino acid residues. However, it is specifically 1 to 20, preferably 1 to 10, more preferably 1 to 5.

The aforementioned substitution is preferably a conservative substitution, which is a neutral mutation causing no functional change. The conservative mutation is a mutation in which substitution occurs among Phe, Trp and Tyr when the substitution site is an aromatic amino acid, among Leu, Ile and Val when the substitution site is a hydrophobic amino acid, between Gln and Asn when the substitution site is a polar amino acid, among Lys, Arg and His when the substitution site is a basic amino acid, between Asp and Glu when the substitution site is an acidic amino acid, and between Ser and Thr when the substitution site is an amino acid having hydroxyl group. More specifically, examples of the conservative substitution include substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Gly, Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr and substitution of Met, Ile or Leu for Val.

Furthermore, a homologous gene may also be used, so long as it has the same function as the target gene. Specifically, the homologous gene should encode a protein that has a homology of 80% or more, preferably 90% or more, more preferably 95% or more, particularly preferably 97% or more, with respect to a sequence of a known protein. Furthermore, since the degeneracy of a gene varies depending on the host into which the gene is introduced, a gene with substitutions of codons that can be easily used in a host may be used. Similarly, so long as the L-threonine producing ability of the target gene can be improved by amplification of the gene, the gene may be extended or shortened at either the N-terminus or C-terminus. The length of the extension or shortening is, for example, 50 or less, preferably 20 or less, more preferably 10 or less, particularly preferably 5 or less, in terms of the number of amino acid residues. More specifically, the protein may have an amino acid sequence which is shortened by 5-50 amino acid residues at either the N-terminus and/or the C-terminus.

A gene homologous to the target gene can be obtained by modifying the nucleotide sequence by, for example, site-specific mutagenesis, so that the encoded protein includes substitutions, deletions, insertions, or additions of amino acid residues at a specific site. Furthermore, such a gene can also be obtained by known mutation treatments, as described below. Examples of the these mutation treatments include a method of treating the nucleotide sequence of the target gene in vitro with hydroxylamine or the like, a method of treating a microorganism having the gene, for example, an Escherichia bacterium, with UV irradiation or a typical mutagenesis agent such as N-methyl-N′-nitro-N-nitrosoguanidine (NTG) or ethyl methanesulfonate (EMS), and a method of introducing a mutation by error-prone PCR. Furthermore, the aforementioned substitutions, deletions, insertions, additions, inversions, or the like, of amino acid residues include naturally occurring mutations (mutant or variant), such as mutations based on individual differences and/or species differences of the microorganism having the target gene. Whether these genes code for a protein that improves the L-threonine producing ability when their expression levels are increased can be confirmed by, for example, introducing these genes into a wild-type strain or a strain having L-threonine producing ability of Escherichia coli and examining whether the L-threonine producing ability is improved.

The target gene may also be DNA that hybridizes with a target nucleotide sequence, or a complementary sequence thereof, or a probe prepared from these sequences under stringent conditions and codes for a protein that improves the L-threonine producing ability of Escherichia bacteria by enhancing the expression. The “stringent conditions” referred to herein include conditions under which a so-called specific hybrid is formed, and a non-specific hybrid is not formed. It is difficult to clearly express this condition by using any numerical value. However, for example, the stringent conditions include conditions under which DNAs having high homology, for example, DNAs having a homology of 50% or more, preferably 60% or more, more preferably 70% or more, more preferably 80% or more, more preferably 90% or more, further preferably 95% or more, most preferably 97% or more, hybridize with each other, but DNAs having a homology lower than the above do not hybridize with each other. Alternatively, the stringent conditions are exemplified by washing one time, preferably 2 to 3 times, under conditions for hybridization at a salt concentration which are typical for washing in Southern hybridization, i.e., 1×SSC, 0.1% SDS at 60° C., preferably 0.1×SSC, 0.1% SDS at 68° C.

Then, a recombinant DNA is prepared by ligating the target gene amplified by PCR to a vector DNA that can function in the cell of a host microorganism. Such a vector includes a vector autonomously replicable in the cell of the host microorganism. Examples of the vector autonomously replicable in Escherichia coli cells include pUC19, pUC18, pHSG299, pHSG399, pHSG398, pACYC184, (pHSG and pACYC are available from Takara Bio Inc.), RSF1010, pBR322, pMW219 (pMW is available from Nippon Gene), and so forth.

A recombinant DNA prepared as described above can be introduced into a microorganism by a known transformation method. Examples include a method of treating recipient cells with calcium chloride so as to increase the permeability of DNA, which has been reported for Escherichia coli K-12 (Mandel, M. and Higa, A., J. Mol. Biol., 53, 159 (1970)), a method of preparing competent cells from cells which are at the growth phase followed by introducing DNA thereinto, which has been reported for Bacillus subtilis (Duncan, C. H., Wilson, G. A. and Young, F. E., Gene, 1, 153 (1977)), and so forth. In addition to these, a method of making DNA-recipient cells into protoplasts or spheroplasts, which can easily take up recombinant DNA, followed by introducing the recombinant DNA into the DNA-recipient cells, which is known to be applicable to Bacillus subtilis, actinomycetes, and yeasts (Chang, S. and Cohen, S. N., Molec. Gen. Genet., 168, 111, 1979; Bibb, M. J., Ward, J. M. and Hopwood, O. A., Nature, 274, 398, 1978; Hinnen, A., Hicks, J. B. and Fink, G. R., Proc. Natl. Sci. USA, 75, 1929 (1978)), may be used.

Increasing the copy number of a gene can also be achieved by introducing multiple copies of the gene into the chromosomal DNA of a microorganism. In order to introduce multiple copies, homologous recombination can be carried out by using a sequence whose multiple copies exist on a chromosomal DNA as targets. As sequences whose multiple copies exist on a chromosomal DNA, repetitive DNA and inverted repeats existing at the end of a transposable element can be used. A target gene may also be introduced into an unnecessary gene on a chromosome by homologous recombination, or a target gene may be introduced into an unnecessary region in tandem. Alternatively, as disclosed in Japanese Patent Laid-open No. 2-109985, it is also possible to incorporate a target gene into a transposon, and allow it to be transferred to introduce multiple copies of the genes into a chromosomal DNA (refer to U.S. Pat. No. 5,595,889). Whether the target gene has been transferred to a chromosome can be confirmed by Southern hybridization using a part of the target gene as a probe.

Furthermore, in addition to increasing the gene copy number, expression of the target gene can be enhanced by replacing an expression control sequence such as a promoter of the target gene on a chromosomal DNA or plasmid with a stronger one, amplifying a regulator that increases the expression, or deleting/attenuating a regulator that decreases the expression as described in International Patent Publication WO00/18935. For example, the lac promoter, trp promoter, trc promoter, Pr promoter derived from lambda phage, and so forth, are known as strong promoters. Furthermore, the promoter of the target gene can be modified to be stronger by introducing a nucleotide substitution into the promoter region. Evaluation of promoter potency, as well as a description of known strong promoters, are set forth in Goldstein et al. (Prokaryotic promoters in biotechnology, Biotechnol. Annu. Rev., 1995, 1, 105-128). Furthermore, it is known that substitution of a spacer between the ribosome binding site (RBS) and the start codon, in particular, substitution of several nucleotides in the sequence immediately upstream from the start codon, has a great impact on mRNA translation efficiency, and these nucleotides can be modified. An expression control region such as a promoter can also be determined by using a promoter search vector or a gene analysis software program such as GENETYX. Expression of the target gene is enhanced by substitution or modification of these promoters. The expression control sequence can be substituted by using, for example, a temperature-sensitive plasmid.

As a result of the increase in activity by such methods, the enzymatic activity increases by at least 10%, 25%, 50%, 100%, 200%, 400%, 600%, or 1000% as compared with a wild-type strain or unmodified strain. Furthermore, these techniques for increasing enzymatic activity can be implemented in combination with each other or in combination with a decrease of an enzymatic activity. Examples of Escherichia bacteria serving as a reference include, as wild-type strains, Escherichia coli W3110 (ATCC 27325), Escherichia coli MG1655 (ATCC 47076), and so forth, which are derived from a prototype wild-type strain K12.

Furthermore, in the bacterium of the present invention, the activity of an enzyme that catalyzes a reaction branching off of the biosynthetic pathway of L-threonine and producing a compound other than L-threonine may be decreased or deficient. Examples of such an enzyme include threonine dehydrogenase, threonine deaminase, and threonine dehydratase. Strains having these enzymes with decreased or deficient activity are described in International Patent Publications WO95/23864, WO96/17930, WO03/080843, WO04/087895, and so forth.

Furthermore, in the bacterium of the present invention, the activity of an enzyme involved in the glycolytic pathway, TCA cycle, or respiration chain that adversely affects the L-threonine production, an enzyme which regulates gene expression, or an enzyme of a byproduct biosynthesis system may be decreased or made deficient, in addition to an L-threonine degradation system enzyme. Examples of a gene coding for such an enzyme include the gene coding for G (sigma) factor of RNA polymerase (rpoS, International Patent Publication WO01/05939 and WO03/074719), phosphoenol pyruvate carboxylase gene (pckA, International Patent Publication WO02/29080), phosphoglucose isomerase gene (pgi, Molecular and General Genetics, 217(1): 126-31 (1989)), pyruvate oxidase gene (poxB, International Patent Publication WO02/29080), and so forth.

In order to decrease or eliminate the activity of an enzyme as described above, a mutation that decreases or eliminates intracellular activity of the enzyme can be introduced into the gene of the aforementioned enzyme on a chromosome by a typical mutation treatment. It is achieved by, for example, deleting a gene coding for an enzyme on a chromosome by gene recombination, modifying an expression control sequence such as a promoter and the Shine-Dalgarno (SD) sequence, and so forth. Furthermore, it can also be achieved by introducing one or more amino acid substitutions (missense mutation), introducing a stop codon (nonsense mutation), introducing a frameshift mutation, which adds or deletes one or two nucleotides, or deleting a part or whole region of a gene (Journal of Biological Chemistry, 272: 8611-8617 (1997)) in a region coding for an enzyme on a chromosome. Furthermore, enzymatic activity can also be decreased or eliminated by constructing a gene coding for a mutant enzyme in which a coding region is deleted, and substituting this gene for a normal gene on a chromosome by homologous recombination or the like, or introducing a transposon or IS factor into the gene. Furthermore, it can also be achieved by inhibiting the expression with an antisense-RNA (Proceedings of the National Academy of Sciences, USA, 95 5511-5515 (1998); Journal of Biological Chemistry, 266, 20833-20839 (1991)).

In order to introduce a mutation that decreases or eliminates the activity of an enzyme as mentioned above by gene recombination, for example, the following method is used. A mutant-type gene can be substituted for a target gene on a chromosome by modifying a partial sequence of the target gene to prepare a mutant gene mutated so that a normal functioning enzyme is not produced, and transforming an Escherichia bacterium with a DNA containing this gene to cause recombination of the mutant gene and the gene on chromosome. Such site-specific mutation utilizing gene substitution by homologous recombination is known, and examples thereof include a method of using a linear DNA, a method of using a plasmid containing a temperature-sensitive replication origin (U.S. Pat. No. 6,303,383; Japanese Patent Laid-open No. 05-007491), and so forth. Furthermore, the aforementioned site-specific mutation by gene substitution utilizing homologous recombination can also be performed by using a plasmid that does not have replication ability in a host.

As a result of decrease in activity or expression by such methods, the enzymatic activity decreases by 75%, 50%, 25%, or 10% as compared with a wild-type strain or unmodified strain, or the activity is completely eliminated. Furthermore, two or more of these methods for decreasing enzyme activity may be implemented in combination, or they may be used in combination with the increase of enzyme activity described above. Examples of an Escherichia bacterium serving as a reference include Escherichia coli W3110 (ATCC 27325), Escherichia coli MG1655 (ATCC 47076), and so forth, which are derived from a prototype wild-type strain K12.

The L-threonine producing bacterium used for the present invention may be a strain which is modified so that it can assimilate sucrose as a carbon source. For example, a strain that assimilates sucrose can be obtained from the E. coli H155 strain by P1 transduction, using its ability to grow with sucrose as the sole carbon source as an index. An L-threonine producing bacterium that can assimilate sucrose as a carbon source can be constructed by referring to International Patent Publication WO90/04636.

Furthermore, in addition to the aforementioned gene manipulation, examples of the method for constructing an L-threonine producing bacterium include a method of treating an Escherichia bacterium by UV irradiation or with a typical mutagenesis agent such as N-methyl-N′-nitro-N-nitrosoguanidine (NTG) and nitrous acid to obtain strains resistant to an L-amino acid or L-amino acid analogue or L-amino acid auxotrophic strains, and selecting a strain which has improved L-threonine producing ability. Examples thereof include the borrelidin resistant mutant strain (U.S. Pat. No. 5,939,307), diaminosuccinic acid resistant mutant strain (International Patent Publication WO00/09661), α-methylserine resistant mutant strain (International Patent Publication WO00/09661), fluoropyruvate resistant mutant strain (International Patent Publication WO00/09661), acetic acid resistant mutant strain (U.S. Pat. No. 5,919,670), threonine resistant mutant strain (International Patent Publication WO90/04636), and AHV resistant mutant strain (Genetika, 16: 206 (1978)).

<3-2> Examples of L-threonine Producing Bacteria that can be Used for the Present Invention

Examples of Escherichia bacteria imparted with an L-threonine producing ability that can be used for the present invention will be described below. However, bacteria that can be used for the present invention are not limited to these examples, so long as the bacterium has an ability to produce L-threonine.

As an example of L-threonine producing bacterium that can be used for the present invention, the Escherichia coli VKPM B-3996 strain (U.S. Pat. No. 5,175,107) is encompassed. This VKPM B-3996 strain was deposited at Russian National Collection of Industrial Microorganisms (VKPM) (Russia, 117545 Moscow, 1 Dorozhny proezd. 1) on Nov. 19, 1987 with a registration number of VKPM B-3996. This VKPM B-3996 strain has the plasmid pVIC40 (International Patent Publication WO90/04636) obtained by inserting the threonine biosynthetic genes (threonine operon thrABC) into pAYC32, which is a broad host vector plasmid having a streptomycin resistance marker (refer to Chistorerdov, A. Y., Tsygankov, Y. D., Plasmid, 1986, 16, 161-167). In pVIC40, feedback inhibition of aspartokinase 1-homoserine dehydrogenase I encoded by thrA in the threonine operon by L-threonine is eliminated.

As an example of L-threonine producing bacterium that can be used for the present invention, the Escherichia coli VKPM B-5318 strain (European Patent No. 0593792) is also encompassed. The VKPM B-5318 strain was deposited at Russian National Collection of Industrial Microorganisms (VKPM) (Russia, 117545 Moscow, 1 Dorozhny proezd. 1) on Nov. 19, 1987 with a registration number of VKPM B-5318. This VKPM B-5318 strain does not require isoleucine, has the temperature-sensitive C1 repressor, PR promoter, and the threonine operon. That is, the threonine biosynthesis-related genes, in which the attenuator region and the native transcription regulating region are deleted, and are located downstream from the gene for the N-terminus region of the Cro protein of lambda phage. Furthermore, this strain contains a recombinant plasmid DNA which has been constructed so that expression of the threonine biosynthesis-involved genes are regulated by the repressor and promoter of lambda phage.

The Escherichia coli 427T23 (U.S. Pat. No. 5,631,157) can also be exemplified as a preferred example of the L-threonine producing bacterium. The 427T23 strain has homoserine dehydrogenase which is not subject to feedback inhibition by L-threonine. Also, this strain has attenuated threonine deaminase activity and can use sucrose as a carbon source. The 427T23 strain has been deposited at American Type Culture Collection with an accession number of ATCC 98082.

The Escherichia coli kat-13 (U.S. Pat. No. 5,175,107) can also be preferably used as the L-threonine producing bacterium. The kat-13 strain is resistant to borrelidin, has homoserine dehydrogenase which is not subject to feedback inhibition by L-threonine, attenuated threonine deaminase activity, and can use sucrose as a carbon source.

The Escherichia coli TDH6 strain (Japanese Patent Laid-open No. 2001-346578) transformed with genes coding for threonine biosynthesis enzymes can also be preferably used as the L-threonine producing bacterium. The TDH6 strain is deficient in threonine dehydrogenase activity, and was obtained by removing pVIC40 that carries the genes coding for the threonine biosynthesis enzymes from the B-3996 strain (U.S. Pat. No. 5,631,157), and has been deposited at Russian National Collection of Industrial Microorganisms (VKPM), (Russia, 117545 Moscow, 1 Dorozhny proezd. 1) with an accession number of VKPM B-3420.

Furthermore, other examples of the L-threonine producing bacterium that can be used for the present invention include the following bacterial strains:

Escherichia coli MG-442 (CMIMB-1628, U.S. Pat. No. 4,278,765)

Escherichia coli VL334/pYN7 (U.S. Pat. No. 4,278,765)

Escherichia coli H-4225 (FERM BP-1236, U.S. Pat. No. 5,017,483)

Escherichia coli H-7256 (FERM BP-2137)

Escherichia coli DSM9807 (KCCM-10168)

### EXAMPLES

The present invention will be explained more specifically with reference to the following examples. However, the present invention is not limited to these examples.

### Example 1

Effect of Regulation of Initially Added Sulfur in Fed-batch Culture

First, the effect of limiting the sulfur in the initial medium of the main culture to a predetermined concentration or lower during L-threonine production was examined in a fed-batch culture.

The VKPM B-5318 strain was cultured on an LB agar medium plate (10 g/L of trypton, 5 g/L of yeast extract, 5 g/L of NaCl, 15 g/L of agar) containing 20 mg/L of streptomycin sulfate at 37° C. for 24 hours, and 1/10 of the cells on the plate were scraped off from one plate and inoculated into 50 mL of LB medium (10 g/L of trypton, 5 g/L of yeast extract, 5 g/L of NaCI) containing 20 mg/L of streptomycin sulfate in a baffle flask to perform a seed culture at 40° C. and 144 rpm for 6 hours.

After completion of the seed culture, the seed culture medium in a volume equivalent to 16% of the main culture medium volume was inoculated into a 1-L jar fermenter charged with 300 mL of the main culture medium, and culture was performed at 40° C. and pH 7.0. The main culture medium composition is shown below.

Main Culture Medium Composition:

| | | |
|---|---|---|
| Sucrose | 27 | g/L |
| Yeast extract | 1.8 | g/L |
| KH₂PO₄ | 1.5 | g/L |
| NaCl | 0.6 | g/L |
| MgSO₄.7H₂O | 0.36 | g/L |
| FeSO₄.7H₂O | 18 | mg/L |
| MnSO₄.4H₂O | 18 | mg/L |
| Streptomycin sulfate | 20 | mg/L |

Ammonium sulfate was appropriately added so that the initially added sulfur content is 0.78 to 0.10 g/L in terms of sulfur content.

The culture was adjusted to pH 7.0 during the culture by adding ammonia gas. After the saccharide in the medium was consumed, 600 g/L of a sucrose aqueous solution was added to perform a fed-batch culture.

After 42 hours of the culture, the L-threonine concentration was determined by HPLC.

As a result, the L-threonine fermentation yield increased with a decrease in sulfur, and it markedly increased, in particular, at a sulfur concentration of 0.29 g/L or lower as shown in Table 1.

TABLE 1

| Initially added | Initially added | Yield |
|---|---|---|
| (NH₄)₂SO₄ (g/L) | S (g/L) | (%) |
| 3.0 | 0.78 | 35.7 |
| 2.0 | 0.54 | 36.6 |
| 1.0 | 0.29 | 38.6 |
| 0.8 | 0.25 | 39.9 |
| 0.4 | 0.15 | 40.8 |
| 0.2 | 0.10 | 43.6 |

### Example 2

Effect of Regulation of Sulfur in the Feed Medium in a Fed-batch Culture

First, in the same manner as in Example 1, the VKPM B-5318 strain was cultured on a LB agar medium plate (10 g/L of trypton, 5 g/L of yeast extract, 5 g/L of NaCl, 15 g/L of agar) containing 20 mg/L of streptomycin sulfate at 37° C. for 24 hours, and 1/10 of the bacterial cells on the plate were scraped off from one plate, and inoculated into 50 mL of LB medium (10 g/L of trypton, 5 g/L of yeast extract, 5 g/L of NaCl) containing 20 mg/L of streptomycin sulfate in a baffle flask to perform seed culture at 40° C. and 144 rpm for 6 hours.

After completion of the seed culture, the seed culture medium in a volume equivalent to 16% of the main culture medium volume was inoculated into a 1-L jar fermenter charged with 300 mL of the main culture medium, and culture was performed at 40° C. and pH 7.0. The main culture medium had the same composition as that used in Example 1.

The culture was adjusted to pH 7.0 during the culture by adding ammonia gas. After the saccharide in the medium was consumed, fermentation was performed with addition of a feed medium containing sucrose, and adjusted so that the sulfur concentration in the fermentation medium is at a predetermined level or lower. The results are shown in Table 2. The sulfur concentration in the feed medium was controlled to be 0 to 1.22 g/L, and the culture was controlled so that the fermentation medium contains 0.144 to 0.548 g/L of sulfur throughout the entire main culture period.

As a result, it was confirmed that the yield of L-threonine was improved also in fed-batch culture by regulating the sulfur concentration in the fermentation medium to 0.35 g/L or lower.

TABLE 2

| (NH₄)₂SO₄ in feed | S in feed | Total S | Yield |
|---|---|---|---|
| solution (g/L) | solution (g/L) | (g/L) | (%) |
| 0.0 | 0.00 | 0.144 | 41.8 |
| 1.0 | 0.24 | 0.225 | 39.1 |
| 2.0 | 0.49 | 0.305 | 38.8 |
| 5.0 | 1.22 | 0.548 | 37.5 |

### INDUSTRIAL APPLICABILITY

According to the present invention, fermentation yield and production of L-threonine can be improved in a method for producing L-threonine by fermentation using a microorganism belonging to the genus Escherichia that has an L-threonine producing ability.

1. A method for producing L-threonine comprising:
A) culturing a microorganism belonging to the genus Escherichia that has an ability to produce L-threonine, in a fermentation medium containing a carbon source, a nitrogen source, and a sulfur source,
B) collecting L-threonine from the culture, wherein the sulfur concentration in the medium is regulated so that it is at a predetermined level or lower.

2. The method according to claim 1, wherein the sulfur concentration in the fermentation medium is regulated so that it is 0.35 g/L or lower.

3. The method according to claim 1, wherein the microorganism is Escherichia coli.

4. The method according to claim 1, wherein an L-threonine biosynthesis enzyme in the microorganism is modified so that the enzyme is not subject to feedback inhibition by L-threonine.

5. The method according to claim 4, wherein the L-threonine biosynthesis enzyme is selected from the group consisting of aspartokinase, homoserine kinase, threonine synthase, and combinations thereof.

6. The method according to claim 1, wherein the sulfur source is selected from the group consisting of sulfates, thiosulfates, sulfites, cysteine, cystine, glutathione, and combinations thereof.

7. The method according to claim 1, wherein the culture method is selected from the group consisting of a batch culture method, a fed-batch culture method, and a continuous culture method.

8. The method according to claim 7, wherein the culture method is a fed-batch culture method or a continuous culture method, and wherein a feed medium containing a sulfur source is added to the culture in a fermenter.

9. The method according to claim 8, wherein said feed medium further contains a carbon source and a nutrient having a growth promoting effect, and wherein said feed medium is added to the culture in the fermenter continuously or intermittently so that the concentration of the carbon source in the culture is maintained at 30 g/L or lower after the end of the logarithmic growth of the microorganism.

10. A method for producing a composition comprising:
A) culturing a microorganism belonging to the genus Escherichia that has an ability to produce L-threonine, in a fermentation medium containing a carbon source, a nitrogen source, and a sulfur source,
B) maintaining the sulfur concentration in the medium during fermentation so that it is at a predetermined level or lower,
C) drying the crude fermentation broth so that the water content is 10% or less by weight.

11. The method according to claim 10, wherein the sulfur concentration in the fermentation medium is regulated so that it is 0.35 g/L or lower.

12. The method according to claim 10, wherein the microorganism is Escherichia coli.

13. The method according to claim 10, wherein an L-threonine biosynthesis enzyme in the microorganism is modified so that the enzyme is not subject to feedback inhibition by L-threonine.

14. The method according to claim 10, wherein the L-threonine biosynthesis enzyme is selected from the group consisting of aspartokinase, homoserine kinase, threonine synthase, and combinations thereof.

15. The method according to claim 10, wherein the sulfur source is selected from the group consisting of sulfates, thiosulfates, sulfites, cysteine, cystine, glutathione, and combinations thereof.

16. The method according to claim 10, wherein the culture method is selected from the group consisting of a batch culture method, a fed-batch culture method, and a continuous culture method.

17. The method according to claim 16, wherein the culture method is a fed-batch culture method or a continuous culture method, and wherein a feed medium containing a sulfur source is added to the culture in a fermenter.

18. The method according to claim 16, wherein said feed medium further contains a carbon source and a nutrient having a growth promoting effect, and wherein said feed medium is added to the culture in the fermenter continuously or intermittently so that the concentration of the carbon source in the culture is maintained at 30 g/L or lower after the end of the logarithmic growth of the microorganism.

19. The method according to claim 10, wherein said composition is an animal feed additive from a fermentation broth.

20. The method according to claim 19, wherein more than 95% L-threonine is included in said feed additive.


SEQUENCE LISTING (6 sequences)

SEQ ID NO: 1 - DNA, 5040 bp, Escherichia coli
  promoter (71)..(99): factor Sigma 70; predicted +1 start at 106
  promoter (104)..(132): factor Sigma 70; predicted +1 start at 139
  promoter (139)..(219): factor Sigma 70; predicted +1 start at 219
  CDS (190)..(255): leader peptide
  attenuator (273)..(307)
  CDS (337)..(2799): thrA
  CDS (2801)..(3733): thrB
  CDS (3734)..(5020): thrC
agcttttcattctgactgcaacgggcaatatgtctctgtgtggattaaaaaaagagtgtctgatagcagcttctgaactggttacctgccgtgagtaaattaaaattttattgacttaggtcactaaatactttaaccaatataggcatagcgcacagacagataaaaattacagagtacacaacatccatgaaacgcattagcaccaccattaccaccaccatcaccattaccacaggtaacggtgcgggctgacgcgtacaggaaacacagaaaaaagcccgcacctgacagtgcgggctttttttttcgaccaaaggtaacgaggtaacaaccatgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtatttccgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctgacatggttaaagtttatgccccggcttccagtgccaatatgagcgtcgggtttgatgtgctcggggcggcggtgacacctgttgatggtgcattgctcggagatgtagtcacggttgaggcggcagagacattcagtctcaacaacctcggacgctttgccgataagctgccgtcagaaccacgggaaaatatcgtttatcagtgctgggagcgtttttgccaggaactgggtaagcaaattccagtggcgatgaccctggaaaagaatatgccgatcggttcgggcttaggctccagtgcctgttcggtggtcgcggcgctgatggcgatgaatgaacactgcggcaagccgcttaatgacactcgtttgctggctttgatgggcgagctggaaggccgtatctccggcagcattcattacgacaacgtggcaccgtgttttctcggtggtatgcagttgatgatcgaagaaaacgacatcatcagccagcaagtgccagggtttgatgagtggctgtgggtgctggcgtatccggggattaaagtctcgacggcagaagccagggctattttaccggcgcagtatcgccgccaggattgcattgcgcacgggcgacatctggcaggcttcattcacgcctgctattcccgtcagcctgagcttgccgcgaagctgatgaaagatgttatcgctgaaccctaccgtgaacggttactgccaggcttccggcaggcgcggcaggcggtcgcggaaatcggcgcggtagcgagcggtatctccggctccggcccgaccttgttcgctctgtgtgacaagccggaaaccgcccagcgcgttgccgactggttgggtaagaactacctgcaaaatcaggaaggttttgttcatatttgccggctggatacggcgggcgcacgagtactggaaaactaaatgaaactctacaatctgaaagatcacaacgagcaggtcagctttgcgcaagccgtaacccaggggttgggcaaaaatcaggggctgttttttccgcacgacctgccggaattcagcctgactgaaattgatgagatgctgaagctggattttgtcacccgcagtgcgaagatcctctcggcgtttattggtgatgaaatcccacaggaaatcctggaagagcgcgtgcgcgcggcgtttgccttcccggctccggtcgccaatgttgaaagcgatgtcggttgtctggaattgttccacgggccaacgctggcatttaaagatttcggcggtcgctttatggcacaaatgctgacccatattgcgggtgataagccagtgaccattctgaccgcgacctccggtgataccggagcggcagtggctcatgctttctacggtttaccgaatgtgaaagtggttatcctctatccacgaggcaaaatcagtccactgcaagaaaaactgttctgtacattgggcggcaatatcgaaactgttgccatcgacggcgatttcgatgcctgtcaggcgctggtgaagcaggcgtttgatgatgaagaactgaaagtggcgctagggttaaactcggctaactcgattaacatcagccgtttgctggcgcagatttgctactactttgaagctgttgcgcagctgccgcaggagacgcgcaaccagctggttgtctcggtgccaagcggaaacttcggcgatttgacggcgggtctgctggcgaagtcactcggtctgccggtgaaacgttttattgctgcgaccaacgtgaacgataccgtgccacgtttcctgcacgacggtcagtggtcacccaaagcgactcaggcgacgttatccaacgcgatggacgtgagtcagccgaacaactggccgcgtgtggaagagttgttccgccgcaaaatctggcaactgaaagagctgggttatgcagccgtggatgatgaaaccacgcaacagacaatgcgtgagttaaaagaactgggctacacttcggagccgcacgctgccgtagcttatcgtgcgctgcgtgatcagttgaatccaggcgaatatggcttgttcctcggcaccgcgcatccggcgaaatttaaagagagcgtggaagcgattctcggtgaaacgttggatctgccaaaagagctggcagaacgtgctgatttacccttgctttcacataatctgcccgccgattttgctgcgttgcgtaaattgatgatgaatcatcagtaaaatctattcattatctcaat

SEQ ID NO: 2 - protein, 21 aa, Escherichia coli [sequence omitted]

SEQ ID NO: 3 - protein, 820 aa, Escherichia coli
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQEAMELSYFGAKVLHPRTITPIAQFQIPCLIKNTGNPQAPGTLIGASRDEDELPVKGISNLNNMAMFSVSGPGMKGMVGMAARVFAAMSRARISVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 4 - protein, 310 aa, Escherichia coli
MVKVYAPASSANMSVGFDVLGAAVTPVDGALLGDVVTVEAAETFSLNNLGRFADKLPSEPRENIVYQCWERFCQELGKQIPVAMTLEKNMPIGSGLGSSACSVVAALMAMNEHCGKPLNDTRLLALMGELEGRISGSIHYDNVAPCFLGGMQLMIEENDIISQQVPGFDEWLWVLAYPGIKVSTAEARAILPAQYRRQDCIAHGRHLAGFIHACYSRQPELAAKLMKDVIAEPYRERLLPGFRQARQAVAEIGAVASGISGSGPTLFALCDKPETAQRVADWLGKNYLQNQEGFVHICRLDTAGARVLEN

SEQ ID NO: 5 - protein, 428 aa, Escherichia coli
MKLYNLKDHNEQVSFAQAVTQGLGKNQGLFFPHDLPEFSLTEIDEMLKLDFVTRSAKILSAFIGDEIPQEILEERVRAAFAFPAPVANVESDVGCLELFHGPTLAFKDFGGRFMAQMLTHIAGDKPVTILTATSGDTGAAVAHAFYGLPNVKVVILYPRGKISPLQEKLFCTLGGNIETVAIDGDFDACQALVKQAFDDEELKVALGLNSANSINISRLLAQICYYFEAVAQLPQETRNQLVVSVPSGNFGDLTAGLLAKSLGLPVKRFIAATNVNDTVPRFLHDGQWSPKATQATLSNAMDVSQPNNWPRVEELFRRKIWQLKELGYAAVDDETTQQTMRELKELGYTSEPHAAVAYRALRDQLNPGEYGLFLGTAHPAKFKESVEAILGETLDLPKELAERADLPLLSHNLPADFAALRKLMMNHQ

SEQ ID NO: 6 - DNA, 66 bp, Escherichia coli
  (1)..(66): leader sequence
atgaaacgcattagcaccaccattaccaccaccatcaccattaccacaggtaacggtgcgggctga